(19) United States
(12) Patent Application Publication
(10) Pub. No.: US 2026/0345370 A1
(43) Pub. Date: Nov. 5, 2026

(54) A DRAINAGE ASSEMBLY

(71) Applicant: SALTS HEALTHCARE LIMITED, West Midlands (GB)

(72) Inventor: Lee Smith, West Midlands (GB)

(21) Appl. No.: 19/108,477

(22) PCT Filed: Sep. 1, 2023
(86) PCT No.: PCT/GB2023/052269

§ 371 Date: Mar. 4, 2025

(30) Foreign Application Priority Data
Sep. 5, 2022 (GB) 2212884.7

(51) Int. Cl. A61F 5/448

(52) U.S. Cl. CPC A61F 5/448

(57) ABSTRACT

A drainage assembly 10 for connecting an ostomy appliance 2 to a secondary collection volume 4, including: a conduit 20 having an entrance and an exit connected by an internal passage 22 for waste to pass through, a structural member 30, which extends along the passage 22, a connector 40 having a body 42, and an anchor formation configured to cooperate with the structural member 30 to inhibit movement of the structural member 30 along the conduit 20.

### FIELD

Embodiments of the invention relate to a drainage assembly. The drainage assembly is primarily for connecting an ostomy appliance to a secondary collection volume.

### BACKGROUND

Ostomy appliances are well known medical devices for users that have a stoma. Generally, the appliance has an opening for the stoma to be received in and an adhesive member that contacts the body and holds the appliance in position. Waste exiting the stoma is collected in a collecting volume, which is formed from two flexible walls connected together. Often the appliance includes an outlet valve, so that a user can empty waste that is collected in the collecting volume without having to remove and dispose of the entire appliance.

At times, a user may want to extend the time between emptying their appliance, for example, when they are sleeping. Typically, a larger capacity bag is used to contain waste while the user is sleeping. In this scenario, the outlet valve is connected to the larger capacity bag by a tube, so that the bag fills with waste.

Embodiments of the present invention seek to alleviate one or more problems associated with the prior art.

### BRIEF DESCRIPTION OF THE INVENTION

According to an aspect of the invention we provide a drainage assembly for connecting an ostomy appliance to a secondary collection volume, including:
a conduit having an entrance and an exit connected by an internal passage for waste to pass through,
a structural member, which extends along the passage,
a connector having a body, and
an anchor formation configured to cooperate with the structural member to inhibit movement of the structural member along the conduit.

The body of the connector may be configured to be inserted in the entrance or exit of the conduit. The anchor formation may engage/contact a part of the structural member to hold the structural member in an axial position. The body may form/provide an outer wall surrounding and/or defining a body passage. The anchor formation may include an opening through the body of the connector.

The structural member may extend through the opening in the body. The structural member may be a filament/threadlike member that extends along a length of the conduit. A length of the structural member may extend through the opening and an end of the structural member may be positioned in the body passage. The end of the structural member may contact an interior surface of the body. The end of the structural member may contact an opposing side of the body to that of the opening.

The opening through the body of the connector may be an open-ended slot/channel which may receive an end of the structural member. The structural member may include a plurality of limbs. Each limb may extend from a substantially central core. A limb may be received in the slot and a pair of limbs may extend outside the body.

The structural member may be positioned between the connector and the conduit. The structural member may be deformed between the conduit and the body. The anchor formation may hold the structural member so as to resist movement under a longitudinal force/pulling.

The connector may provide a substantially permanent connection to a night drainage bag. The connector may provide a removable connection to an ostomy appliance and/or a night drainage bag.

### BRIEF DESCRIPTION OF THE FIGURES

In order that the present disclosure may be more readily understood, preferable embodiments thereof will now be described, by way of example only, with reference to the accompanying drawings, in which:

FIG. 1 is a schematic representation of a drainage assembly,

FIG. 2 is a perspective illustration of a drainage assembly,

FIG. 3 is a cross-sectional side view of the drainage assembly of FIG. 2,

FIG. 4 is a cross-sectional through A-A of FIG. 2,

FIG. 5 is a perspective illustration of an alternative drainage assembly,

FIG. 6 is a cross-sectional schematic view through B-B of FIG. 5, and

FIG. 7 is a perspective schematic of the alternative drainage assembly.

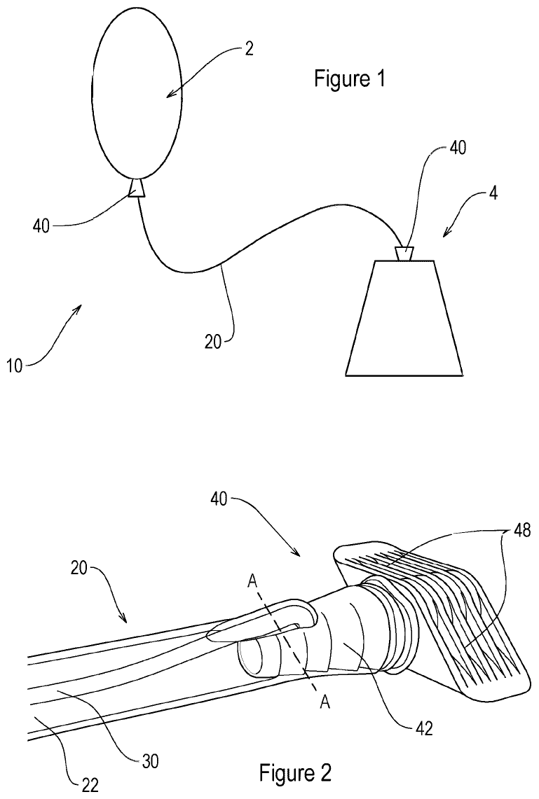

### DETAILED DESCRIPTION OF THE DISCLOSURE

Referring to the figures, a drainage assembly 10 for connecting an ostomy appliance 2 to a secondary collection volume 4 (or night drainage bag) is illustrated. The drainage assembly 10 includes a conduit 20, a structural member 30 and a connector 40.

The conduit 20 has an entrance and an exit. In other words, the conduit 20 has an opening at one end that forms the entrance and an opening at an opposing end that forms the exit. The entrance and the exit are connected by a passage 22. The passage 22 forms an internal flow path for waste to pass through. In use, when the drainage assembly 10 is connected to the ostomy appliance 2 and the secondary collection volume 4, waste is able to flow from the ostomy appliance 2 through the conduit 20 to the secondary collection volume 4.

In embodiments, the conduit 20 is between around 1 and 1.5 metres long (a preferred length is around 1.2 metres) but it should be appreciated that this length can be altered as desired. Usually, the drainage assembly 10 will be used at night to allow an ostomy appliance 2 (which is connected to a user about their stoma) to connect to a secondary collection volume 4. In other words, a collection volume of an ostomy appliance 2 is connected to another collection volume 4, to reduce the need for the user to get up in the night to empty their appliance. Thus, the conduit 20 will be long enough to extend between an appliance 2 attached to the user to the secondary collection volume 4 that could be on the ground surface or some distance away from the appliance 2.

In the present example, the conduit 20 is formed from a plastics material—for example, one of a thermoplastic elastomer (TPE), a thermoplastic polyurethane (TPU) or a rubber-like material may be suitable.

The structural member 30 extends along the passage 22. The structural member 30 acts within the passage to prevent or at least reduce kinking of the conduit 20. Preferably, the structural member 30 extends along substantially the entire length of the conduit 20 but this isn't necessarily the case. For example, in use, some sections of the conduit 20 may be more prone to kinking or blocking than others (i.e. a length of the conduit 20 that is close to the user/extending across a bed surface or similar and/or that is more likely to have weight placed on it) and, as such, the structural member 30 may extend along a portion of the conduit 20.

The connector 40 includes a body 42 and an anchor formation. In some embodiments, the body 42 is configured to be inserted in the entrance or exit of the conduit 20 (as can be seen by the figures). It should be appreciated that the body 42 engages/contacts the conduit 20 to provide a sealed connection. In the illustrated examples, the body 42 forms an outer wall that surrounds and/or defines a body passage.

In some embodiments, the connector 40 may be held in the entrance/exit of the conduit 20 via a friction fit (and/or adhesive to hold the connector 40 in place). The body 42 may include one or more frustoconical portions that gradually increase in size (i.e. the narrowest portion of the body 42 extends furthest into the conduit 20 and the widest may be positioned adjacent or at the entrance / exit).

In some embodiments, the connector 40 provides a substantially permanent connection to a night drainage bag or collection volume 4. This example is shown in the figures. The connector 40 includes tapering portions 48 on either side of the body passage that provide a surface for welding or otherwise connecting the connector 40 to a flexible film layer of a night drainage bag. Alternatively, the connector 40 provides a removable connection to an ostomy appliance 2 and/or a night drainage bag/collection volume 4. This example is not illustrated but would include a connection mechanism that cooperates with a valve fitting.

The anchor formation is configured to cooperate with the structural member 30 to inhibit movement of the structural member 30 along the conduit 20. In embodiments, the anchor formation engages / contacts a part of the structural member 30 to hold the structural member 30 in an axial position. In other words, the structural member 30 is held in position relative to the conduit 20 by the anchor formation.

In some embodiments, the anchor formation includes an opening through the body 42 of the connector 40 (e.g. the structural member 30 extends through the opening). In other words, the structural member 30 may engage with a recess/aperture or other formation in the body 42 which holds the structural member 30 in position and inhibits longitudinal movement of the structural member 30 along the conduit 20.

In embodiments (for example, see FIGS. 2 to 4), the structural member 30 is a filament or threadlike member (i.e. a long thin device) that extends along a length of the conduit 20. As mentioned above, the structural member 30 may extend along substantially the entire length of the conduit 20. In this example, the filament could oscillate from side to side of the passage 22, so it may not be exactly the same length as the conduit 20 itself.

In embodiments, the filament is made from a material that resists kinking and/or is difficult to deform (i.e. it does not easily deform from a generally circular thread when put under pressure (e.g. a person's weight)). Suitable materials may include one of a TPE, TPU or rubber-like polymer selected to reinforce the conduit 20 shape, so that the passage 22 is maintained in an open condition at all times. A preferred material is polypropylene as it is flexible enough to bend with the conduit 20 but rigid enough to resist kinking and collapsing of the passage 22, as discussed above.

It should be appreciated that although the filament illustrated in FIGS. 2 to 4 is a generally circular thread that this does not necessarily need to be the case. The filament could have an alternative cross-section (e.g. square, diamond or triangular, etc.) and still perform in a similar way to a circular filament—the important aspect is that the filament is thin and can be bent such that is extends through the required portion of the connector 40 (see below).

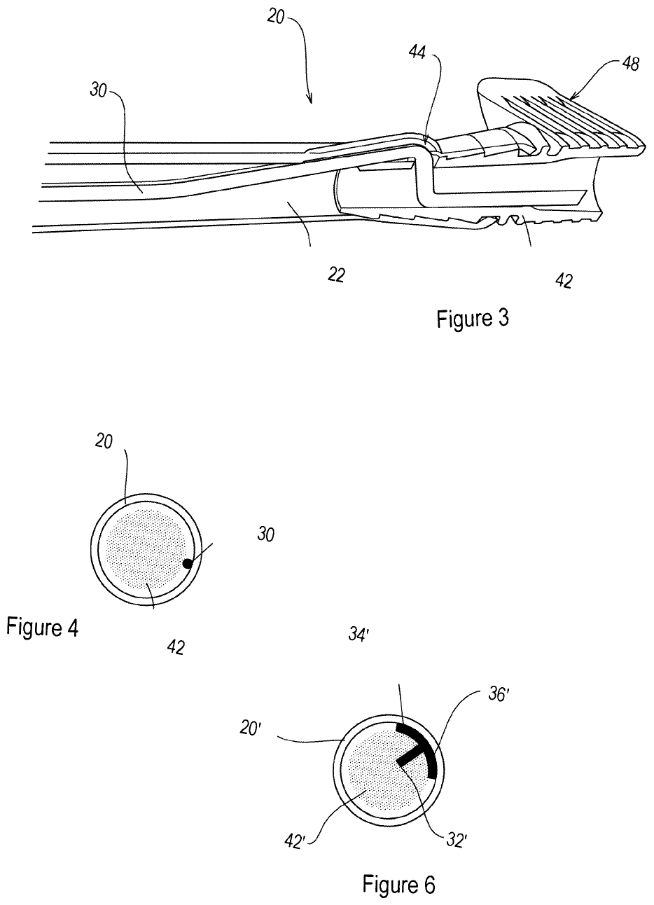

Referring particularly to FIG. 3, the opening in the body is provided by aperture 44. A length of the structural member 30 extends through the aperture 44. In other words, the filament extends around over the outside of the body 40 and through the aperture 44 to the inside of the body 42. The body 40 and the conduit 20 exert a force on the structural member 30 (which is in between) and, thus, it is held against the body 40.

In the present example, an end of the structural member 30 is passed through the aperture 44 which result in the structural member 30 being positioned in the body passage. More specifically, the end of the structural member 30 contacts an interior surface of the body 40 (and lies against the interior surface of the body 40—as shown in FIG. 3).

In embodiments (see FIG. 3, for example), the end of the structural member 30 contacts an opposing side of the body 42 to that of the opening/aperture 44. In other words, the filament extends through the aperture 44 on a first side of the body 40 then, once in the body passage, the filament extends across the passage and is located against the side opposite of the aperture 44.

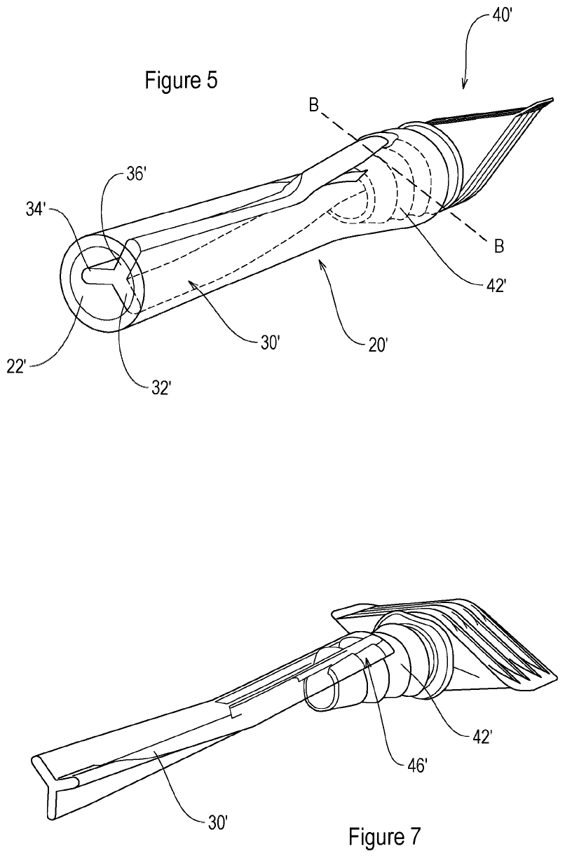

A second configuration of structural member and connector is illustrated in FIGS. 5 to 7. Where features are similar or the same as those already described, they are allocated the same reference number with a prime symbol (i.e. connector 40 becomes connector 40′). Those features that are new have a new number reference and still include the prime symbol to illustrate that it is part of this second configuration. It should be appreciated that the features already described above could apply to this second configuration—unless conflicting, it should be appreciated that any features may be combined.

In embodiments (for example, FIGS. 5 to 7), the structural member 30′ has a plurality of limbs that extend outwards from a central core. In other words, the structural member 30′ forms a star shape in cross-section. The illustrated example is a three-pointed star (and has a first limb 32′, a second limb 34′ and a third limb 36′). Although it should be appreciated that alternative shapes could be used as desired.

In embodiments (see particularly FIG. 7), the opening in the body 40′ is an open-ended slot / channel 46′. An end of the structural member 30′ is received in the channel 46′. More specifically, the first limb 32′ is located in the channel 46′—the end of the structural member 30′ abuts the end of the channel 46′.

The second and third limbs 34′, 36′ extend outside/above the channel 46′. The conduit 20′ forces the second and third limbs 34′, 36′ to deform and lie flat against the outer surface of the body 42′ (shown in the cross-section of FIG. 6). Similarly to the configuration described above in relation to FIGS. 2 to 4, the conduit 20′ and body 42′ exert forces on the structural member 30′ to hold it in position.

In some embodiments, the structural member 30, 30′ (or at least a portion of the structural member 30, 30′) is positioned between the connector 40, 40′ and the conduit 20, 20′. It should be appreciated that the anchor formation does not necessarily have to rely on this positioning since the structural member 30, 30′ could cooperate with a different formation that holds it in position.

In embodiments, the conduit 20, 20′ fits tightly over the body 42, 42′ of the connector 40, 40′. This means that using an anchor formation that includes either deforming or pinning the structural member 30′ between the conduit 20, 20′ and the body 42, 42′ provides a reliable way of holding the structural member 30, 30′ in position. This allows the anchor formation to hold the structural member 30, 30′ so as to resist movement under a longitudinal force/pulling. This can be particularly advantageous as the conduit 20, 20′ may be more elastic than that of the structural member 30, 30′, so if a user accidentally pulls the conduit 20, 20′, and there is no anchor mechanism, the structural member 30, 30′ could inadvertently migrate along the passage 22, 22′ and stop working as intended.

When used in this specification and claims, the terms “comprises” and “comprising” and variations thereof mean that the specified features, steps or integers are included. The terms are not to be interpreted to exclude the presence of other features, steps or components.

The invention may also broadly consist in the parts, elements, steps, examples and/or features referred to or indicated in the specification individually or collectively in any and all combinations of two or more said parts, elements, steps, examples and/or features. In particular, one or more features in any of the embodiments described herein may be combined with one or more features from any other embodiment(s) described herein.

Protection may be sought for any features disclosed in any one or more published documents referenced herein in combination with the present disclosure.

Although certain example embodiments of the invention have been described, the scope of the appended claims is not intended to be limited solely to these embodiments. The claims are to be construed literally, purposively, and/or to encompass equivalents.

1. A drainage assembly for connecting an ostomy appliance to a secondary collection volume, comprising:
a conduit having an entrance and an exit connected by an internal passage for waste to pass through,
a structural member, which extends along the passage,
a connector having a body, and
an anchor formation configured to cooperate with the structural member to inhibit movement of the structural member along the conduit.

2. A drainage assembly according to claim 1 wherein the body of the connector is configured to be inserted in the entrance or exit of the conduit.

3. A drainage assembly according to claim 1 wherein the anchor formation engages/contacts a part of the structural member to hold the structural member in an axial position.

4. A drainage assembly according to claim 1 wherein the body forms an outer wall surrounding and/or defining a body passage.

5. A drainage assembly according to claim 1 wherein the anchor formation includes an opening through the body of the connector.

6. A drainage assembly according to claim 5 wherein the structural member extends through the opening in the body.

7. A drainage assembly according to claim 6 wherein the structural member is a filament/threadlike member that extends along a length of the conduit.

8. A drainage assembly according to claim 6 wherein a length of the structural member extends through the opening and an end of the structural member is positioned in the body passage.

9. A drainage assembly according to claim 8 wherein the end of the structural member contacts an interior surface of the body.

10. A drainage assembly according to claim 9 wherein the end of the structural member contacts an opposing side of the body to that of the opening.

11. A drainage assembly according to claim 5 wherein the opening through the body of the connector is an open-ended slot/channel which receives an end of the structural member

12. A drainage assembly according to claim 11 wherein the structural member includes a plurality of limbs.

13. A drainage assembly according to claim 12 wherein each limb extends from a substantially central core.

14. A drainage assembly according to claim 12 wherein a limb is received in the slot and a pair of limbs extend outside the body.

15. A drainage assembly according to claim 1 wherein the structural member is positioned between the connector and the conduit.

16. A drainage assembly according to claim 15 wherein the structural member is deformed between the conduit and the body.

17. A drainage assembly according to claim 1 wherein the anchor formation holds the structural member so as to resist movement under a longitudinal force/pulling.

18. A drainage assembly according to claim 1 wherein the connector provides a substantially permanent connection to a night drainage bag.

19. A drainage assembly according to claim 1 wherein the connector provides a removable connection to an ostomy appliance and/or a night drainage bag.

20. A connector for a drainage assembly according to claim 1 including a body configured to be inserted in the entrance or exit of a conduit, and an anchor formation configured to cooperate with a structural member to inhibit movement of the structural member along the conduit.
